(19) United States
(12) Patent Application Publication
(10) Pub. No.: US 2008/0172002 A1
(43) Pub. Date: Jul. 17, 2008

(54) FLEXIBLE VALVE FOR BLOOD TREATMENT SET

(76) Inventors: David Bell, Grayslake, IL (US); William J. Schnell, Libertyville, IL (US); David S. Utterberg, Seattle, WA (US)

Correspondence Address: Garrettson Ellis;Seyfarth Shaw LLP, Suite 4200, 55 East Monroe Street, Chicago, IL, 60603, US

(21) Appl. No.: 12/053,691

(22) Filed: Mar. 24, 2008

Related U.S. Application Data
(63) Continuation of application No. 11/270,105, filed on Nov. 9, 2005, now abandoned.

(51) Int. Cl. A61M 39/22; F16K 5/00

(52) U.S. Cl. 604/248; 251/314

(57) ABSTRACT

A flexible valve for a blood treatment set. A flexible fitting including a first chamber, a second chamber, and a slit barrier connecting the first chamber and second chamber and positioned therebetween. An actuating member is positioned in rotating engagement with the slit barrier that is moveable from a first rotational position, in which the slit is closed, to a second position, in which the slit is open. The flexible valve is attachable to a port of a blood treatment chamber. A user opens the valve by rotating the actuating member from a first position to a second position and closes the valve by rotating the valve from the second position to a first position.

### CROSS-REFERENCE TO RELATED APPLICATION(S)

This application is a continuation of U.S. Ser. No. 11/270,105, filed Nov. 9, 2005, entitled “Flexible Valve for Blood Treatment Set.

### BACKGROUND OF THE INVENTION

Blood treatment involves removing blood from a patient, treating the blood, and then directing treated blood, or a component thereof, back to the patient. There are many types of blood treatment sets. Some sets include arterial and venous lines. Other sets include a shared line that handles both treated and untreated blood both to and from the body.

The largest number of blood treatment sets in use today are those having membranes in the flow treatment device. Such treatment devices include hemodialysis units, plasmapheresis units, hemofiltration units, and membrane-type blood oxygenators for open heart surgery. Also included are bubble-type oxygenators and other, more exotic blood treatment devices, in which the blood passes across a unit carrying a fixed bed of enzyme or other bioactive agent for various forms of blood treatment, which are at the present time largely experimental. While most references herein will be directed to hemodialyzers, it is to be understood that other flow-through blood treatment devices are intended for modification in accordance with this invention.

The circulation of blood is a complex process requiring a number of parts connected together at various points to form a complete system. For example, in hemodialysis, a membrane dialyzer is attached to a hemodialyzer hardware unit for providing dialysis solution, and controlling the parameters of blood flow through the membrane dialyzer. The dialyzer is connected at its respective arterial and venous blood flow ports with an arterial set and a venous set at one end of each, while the arterial and venous sets are connected to typically the fistula of a patient at the other set ends, to provide the circulatory flow path of blood from the patient, through the dialyzer, and back to the patient. Such arterial and venous sets carry connected branch lines, which connect the set to various important ancillary functions of the dialysis operation. One of the branch connection lines connects with a source of anticoagulant such as heparin. This source typically comprises a syringe which may be controlled by the dialyzer hardware unit to provide a proper heparinization of the blood, to prevent clotting in the dialyzer or other blood treatment unit. Other branch lines may connect with one or more pressure monitors. Another branch line from one of the sets typically connects with a container of intravenous quality saline solution for priming of the set, flushing it out, and for the emergency addition of saline to the patient in the event of a crisis brought onto the patient by excessive ultrafiltration. Also, the sets may have branch tubing extending from a blood chamber of the sets to which a syringe may be connected, to add or remove air to adjust the blood level in the chamber. Accordingly, a blood treatment set requires a number of connection points and valves to properly function.

There are, however, drawbacks to the existing technology used for these connection points. For instance, when a large, rigid syringe or the like is attached to blood chamber using a rigid, needless valve, the syringe can act as a large lever arm. If the syringe is jostled, struck, or moved accidentally, the syringe may act as a lever and snap the valve from the blood treatment set, thereby damaging the set and causing blood to leak from the system. Accordingly, what is needed is a valve that is engineered to prevent such a mishap.

### SUMMARY OF THE INVENTION

In one aspect the invention comprises a medical valve. The medical valve includes a first chamber, a second chamber, and a flexible barrier with a slit connecting the first chamber and the second chamber, and positioned therebetween. An actuating member is in rotating engagement with the flexible barrier. The actuating member is moveable between a first position, in which the slit is closed, and a second position in which the slit is open. The flexible barrier may be part of a flexible fitting, preferably made of an elastomer.

In another aspect, the invention comprises a system including a blood treatment chamber having at least one rigid access port, and a medical valve providing access to the blood treatment chamber and connected to the port. The medical valve includes a flexible barrier attached to the rigid port, and an actuating member rotationally engaged with the flexible barrier.

In a further aspect, the invention comprises a method of controlling fluid communication into a blood treatment chamber. A medical valve including a flexible barrier and an actuating member is attached to a rigid access port on the chamber. The actuating member is rotated to compress the flexible barrier such that the fluid can flow through an opening in the barrier and through the medical valve.

The flexible fitting may comprise, in some embodiments, a first end including a female port for engagement with a male connector of a blood flow tubing assembly, and a second end for engagement with a rigid port of a blood treatment chamber. Alternatively, any known type of connection for a blood treatment chamber may be used.

Also, the first end portion of the flexible fitting may have a threaded end for engagement with a corresponding threaded portion of a mating flow connector such as a male luer lock connector. Thus, the threaded end is made of flexible material, providing ease of manufacture with good, sealing threaded retention.

The actuating member may comprise a ring portion having an aperture with the flexible barrier being positioned within the aperture. In some embodiments, the aperture may comprise a generally rectangular portion having opposed, arcuate ends, in which opposed sides of the aperture act as cams to compress the flexible barrier when the actuating member is in the second position, to cause the slit to open and to allow fluid communication between the first and second chambers.

The actuating member may also have a handle attached to the ring portion, and a cylindrical portion attached coaxially to the ring portion, wherein the cylindrical portion may be shaped and adapted for sliding engagement with a rigid port of a medical device chamber.

Thus, the opposing sides act as cams which force open the slit of the barrier such that fluid can flow through the valve when the actuating member is in its second position, and the opposing sides force closed the slit of the barrier such that fluid cannot flow through the valve when the actuating member is in the first position.

Also, a method of controlling fluid communication into a blood treatment chamber is provided, in which the method comprises: attaching a medical valve including the flexible, slit barrier and an actuating member to a blood treatment chamber; attaching a fluid flow connector to the medical valve; and rotating the actuating member to compress the slit barrier and open the slit such that fluid can flow through the medical valve. This is accomplished by radial compression of the slit barrier in a direction longitudinal of the direction of extension of the slit.

Additionally, by this invention the valve includes a tubular, flexible fitting made entirely of elastomer, having a frustoconical lumen like a luer if desired, but alternatively with a cylindrical lumen or a countertapered lumen, and optionally a threaded end or lugs for connection with a male luer lock connector or the like. This threaded end can thus be molded in place as part of the flexible fitting as described herein. Alternatively, the flexible fitting may occupy other known positions for a female luer connector in other contexts and types of apparatus, in which the flexible fitting is a molded component as part of other components such as T-connector, chambers, or the like, in which a cylindrical or tapered end lumen and a threaded member or lugs can be provided in a generally conventional manner except that the material used is elastomeric. Also, a typically perforated, flexible flow barrier may occupy the lumen of the flexible fitting.

### BRIEF DESCRIPTION OF DRAWINGS

FIG. 1 is an exploded, elevational view of a portion of a blood treatment set utilizing the medical valve of one embodiment of the invention.

FIG. 2 is a perspective view of the medical valve shown in FIG. 1.

FIG. 3 is a sectional view taken along line 3-3 of FIG. 2.

FIG. 4A is a sectional view along line 4-4 of FIG. 2, with an actuating member positioned such that the valve is open.

FIG. 4B is a similar sectional view of the medical valve of FIG. 2, with the actuating member positioned such that the valve is closed.

FIG. 5 is a perspective view of a cap, used in a blood treatment set, with the medical valve of FIG. 1 shown without the actuating member attached thereto.

FIG. 6 is a partial sectional, partial elevational view of the cap of FIG. 5.

FIG. 7 is a perspective view of the cap of FIG. 5 with the actuating member attached and shown in an open position in full lines and a closed position in broken lines.

FIG. 8 is an elevational view of another embodiment of a tubular flexible fitting which may be used as a substitute for the flexible fitting disclosed in the previous drawings.

FIG. 9 is a sectional view taken along line 9-9 of FIG. 8.

### DETAILED DESCRIPTION

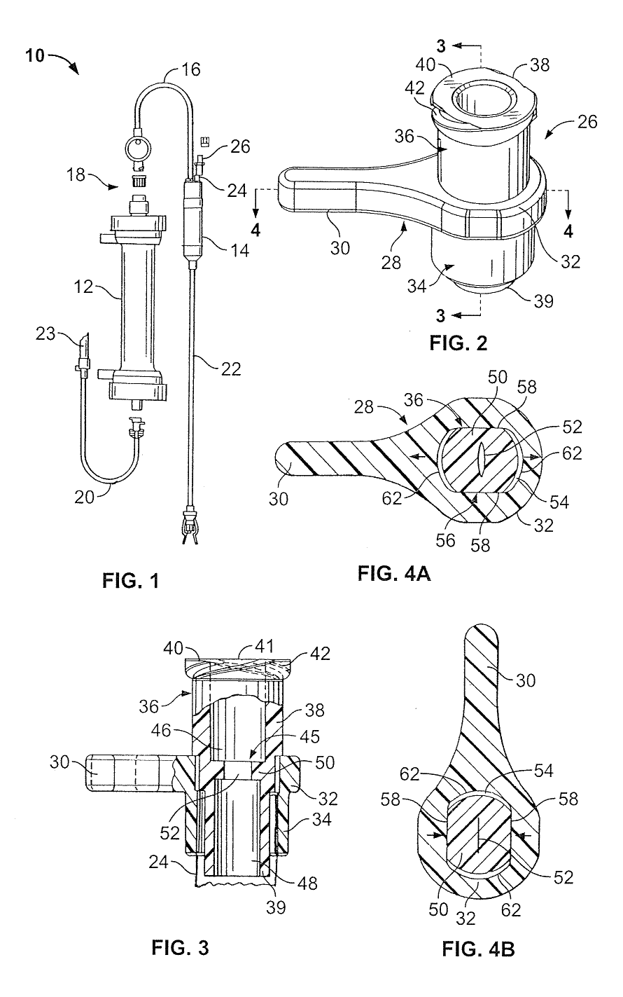

Referring to FIG. 1, a portion of a blood treatment set 10 is shown for illustrative purposes. The blood treatment set includes components, such as a hemodialyzer 12 and a blood treatment chamber, e.g., venous chamber 14. Blood flow tubing 16 attaches hemodialyzer 12 to venous chamber 14 through connector 18. Additional blood flow tubing 20, 22, and pump tubing 23, is present to connect hemodialyzer 12 and venous chamber 14 to other portions of the blood treatment set 10. Venous chamber 14 carries a port tube 24 to connect venous chamber 14 to another part of the treatment set, such as a syringe access site or a pressure monitor line. Port 24 is attached to valve 26 in telescoping manner. Valve 26 is used as an interface to connect venous chamber 14 to other connections.

The previous components (i.e. hemodialyzer 12 and venous chamber 14) are provided for illustrative purposes as a means to describe an exemplary environment in which valve 26 operates. A number of other components and a number of different blood treatment configurations can utilize valve 26 without departing from the principles set forth herein. Specifically, a “blood treatment chamber” may comprise blood flow tubing or any enlargement in the flow path thereof.

Referring to FIG. 2, valve 26 includes an actuating member 28 and tubular, flexible fitting 36. In some embodiments, actuating member 28 is formed from a rigid material, such as a plastic. Actuating member 28 comprises a handle arm 30, a ring portion 32, and cylindrical portion 34. The ring portion 32 surrounds flexible barrier 50 defined by fitting 36, and is utilized to open and close valve 26 as will be discussed herein. The handle arm 30 provides an interface for a user to grasp when moving actuating member 28 from a first position (FIG. 4B), in which valve 26 is closed, to a second position (FIG. 4A), in which valve 26 is open, and vice versa. The principles of operation for valve 26 will be further discussed herein.

Fitting 36 is made from a flexible material, such as polyurethane and comprises flexible barrier 50. An exemplary material for fitting 36 is Laripur 8025, made by Coim SpA, a European company. Fitting 36 in one example has a first, upper, tubular end portion 38 and a second, lower, tubular end portion 39. First end portion 38 in one example includes a female connector 40 with a threaded portion 42 at its mouth 41 for engagement with a threaded portion of a male connector. In one example, female connector 40 could have a tapered lumen shaped and adapted to receive a male luer lock connector.

Referring still to FIG. 3, second end portion 39 of fitting 36 is shaped and dimensioned for insertion into or around inlet port 24 of a component of blood treatment system 10 such as chamber 14. The interior 45 of fitting 36 includes a first chamber 46, a second chamber 48, and a flexible barrier 50 positioned between first chamber 46 and second chamber 48 to block flow between the chambers. Flexible barrier 50 includes a slit 52 (FIGS. 4A and 4B) that extends through barrier 50 from first chamber 46 to second chamber 48. The actuating member 28 is positioned such that ring portion 32 surrounds the area around flexible barrier 50. Cylindrical portion 34 extends downward in a coaxial manner from ring portion 32. Cylindrical portion 34, in one example, is shaped and dimensioned to rotate freely around rigid port 24, as will be further discussed herein.

Referring to FIG. 4A, ring portion 32 of actuating member 28 includes aperture 54 through which fitting 36 is inserted. Aperture 54 of ring portion 32 is defined by walls to include in cross section a rectangular portion 56, having two opposed straight sides 58 and two opposed, arcuate, convex ends 62. Thus, one dimension of recess 54 is terminated by flat walls 58 and another dimension of recess 54 is terminated by concave walls 62, the latter dimension being the larger.

Flexible barrier 50 itself may, in its undistorted configuration, be circular in cross sectional shape and of a diameter which is greater than the dimension between flat walls 58, and less than the dimension between arcuate walls 62, permitting it to achieve the distorted shapes imposed upon it by actuating member 28 in the respective positions of FIGS. 4A and 4B. Because of this distortion, in the position of FIG. 4A, actuating member 28 forces slit 52 into an open position. In FIG. 4B, a 90° degree rotation of actuating member 28 forces slit 52 into a closed position. In some embodiments, slit 52 is held with a measure of pressure in that closed position to provide a good seal. Alternatively, sealing barrier 50 may have an oval, rectangular or other cross-sectional shape.

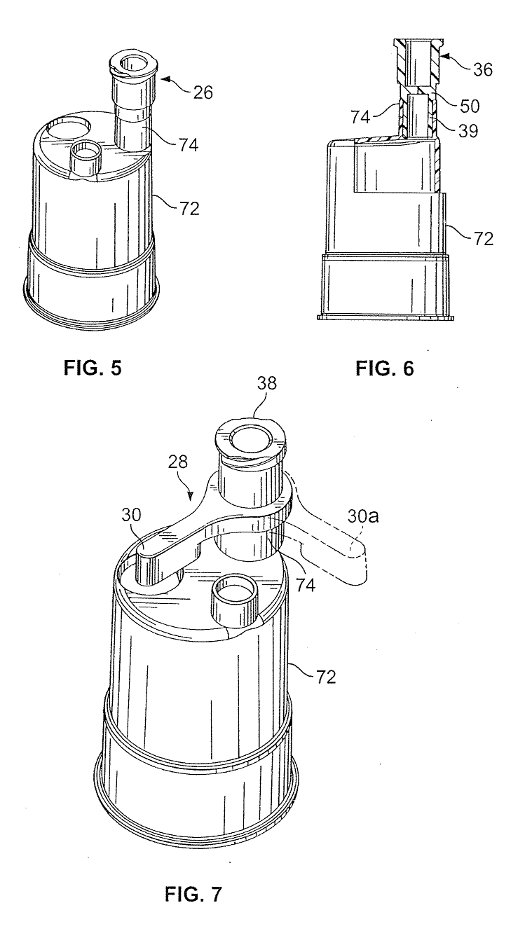

Referring to FIGS. 5 and 6, an exemplary implementation of valve 26 is shown for illustrative purposes. FIG. 5 shows valve 26 attached to a conventional cap 72 for a chamber. The valve 26 is shown with the actuating member 28 removed for ease of reference. In one example, cap 72 is made of rigid plastic, and used to provide one or more ports to a blood treatment chamber. Valve 26 is shown in FIG. 6 attached to a rigid port 74 that is molded onto cap 72. Second end portion 39 of fitting 36 is inserted into rigid port 74, and bonded to an interior portion of port 74. If fitting 36 is made of polyurethane, it will be usually desirable to use a solvent bond to bond fitting to rigid port 74. If another material is used for fitting 38, then bonding can be carried out through use of another mechanism, such as adhesive. The actuating member 28 is positioned as described, such that its cylindrical portion 34 rotates freely about port 74, and sealing barrier 50 can be radially inwardly compressed.

Referring to FIG. 7, it can be seen that to provide fluid communication with the interior of cap 72, the handle arm 30 of actuating member 28 is rotated from the first position, in which slit 52 is closed, to the second position 30a, in which the slit 52 is open. In use, one could connect a large syringe or other connector into fitting 38 and turn arm 30 to open valve 26, thereby allowing fluid communication with the chamber to which cap 72 is connected. If, for whatever reason, someone or something were to make contact with the blood treatment set, thereby jostling or moving fitting 38 and whatever is connected to it, the fitting 38 will bend, because of its flexible nature. Accordingly, the valve will not snap away from port 74, and a mishap will be prevented.

In the above embodiment, the actuating member 28 provides radial pressure to flexible barrier 50 to force slit 52 into an open position, or a closed one, depending upon the rotational position of actuating member 28. As stated, a polyurethane material may be used as one example. Other examples of materials which are suitable are other, known, solvent bondable elastomers which can thus be easily connected to tubing at the respective end portions 39, 41 of fitting 36. However, solvent bondable materials typically do not have a high elastic memory, so actuating member 28 is proportioned to provide radial pressure for both opening and closing of slit 52.

In other embodiments, the elastomeric material used for tubular, flexible fitting 36 can be a high elastic memory thermoplastic or thermoset elastomer, such as many silicone or isoprene elastomers. With such a high memory material, actuating member 28 could positively open a normal, closed slit with radial pressure as in this invention, but in another position the actuator would allow the materials memory to reclose the slit, using the natural and spontaneous elastic memory of the material. This might be accomplished to through the use of a flexible barrier 50 which is of rectangular cross section and of less width in one transverse dimension than in the other transverse dimension.

Alternatively, with such a material having high elastic memory, actuating member 28 could positively close with radial pressure naturally open slit, but in another position, it would allow the memory of the elastomer to spontaneously reopen the slit by the absence of radial closing pressure.

Typically, such high elastic memory elastomers are not effectively solvent bondable, so, typically, other means for attaching tubing to such elastomer fittings would generally be needed.

Typically, fitting 36, in some embodiments comprises an unitarily molded part including first upper tubular end portion 38, and second, lower, tubular end portion 39, plus flexible sealing barrier 50. Slit 52 can then be molded into this unitary part, for example being molded in an open position that is subsequently closed when fitted into actuating member 28.

As another alternative, such a fitting 36 has a slit that closes by its own accord utilizing the elastic memory of fitting 36. In this situation, actuating member might not be needed, and with such a self-sealing slit, the valve could be opened by applying radially inward, opposed finger pressure outside of the valve along the axis of the slit.

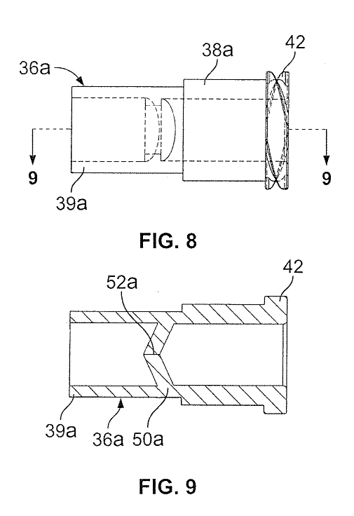

Referring to FIGS. 8 and 9, another design of fitting 36a is shown comprising, as in the previous embodiment, an elastomeric material which preferably may have a significant amount of elastic memory fitting 36a, comprises, as before, first tubular end portion 38a and second tubular end portion 39a with flexible barrier 50a positioned within the lumen of the device. In this embodiment, flexible barrier 50a, having slit 52a as in the previous embodiment, is of a conical shape extending toward the end of second end portion 39a so that fluid flow can take place toward the left through actuating member 36a as shown in FIG. 9, but fluid flow takes place towards the right much less easily or not at all, because flexible barrier 50a acts as a one way valve due to its conical nature. As in the previous embodiment, first end portion 38a carries a threaded portion 42 which is an integral part of the molded, flexible fitting 36a, to engage and lock threads and the like of a male connector. Alternatively, threads 42 may be lugs that are engaged a threaded sleeve of a male connector.

While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

That which is claimed:

1. A medical valve device for use with a blood treatment set, the device comprising:
a tubular fitting including:
first and second chambers,
a first end portion for engagement and communication with a first port, the first end portion communicating with the first chamber,
a second end portion for engagement and communication with a second port, the second end portion communicating with the second chamber, and
a generally central portion having an internal flexible barrier having a slit extending between the first chamber and the second chamber; and
an actuating member in rotatable engagement radially around the fitting central portion and including:
an exterior portion for manual movement of the actuating member, and
an inner surface generally in contact with the fitting central portion, the inner surface including at least a first flat,
wherein the actuating member is moveable to a first position whereby the first flat is positioned relative to the slit to compress the fitting central portion and open the slit to permit communication between the first and second chambers, and the actuating member is moveable to a second position whereby the first flat is positioned relative to the slit to compress the fitting central portion to close the slit to prevent communication between the first and second chambers.

2. The device of claim 1 wherein actuating member includes a second flat generally opposed to the first flat, the two flats cooperating in the first and second positions to open and close, respectively, the slit.

3. The device of claim 2 wherein the flats are joined by opposed arcuate surface portions, the flat surface portions acting as cams to compress the fitting central portion.

4. The device of claim 1 wherein the actuating member exterior portion includes a handle portion for manual actuating thereof.

5. The device of claim 1 wherein the tubular fitting is a unitary member formed integral and made of elastomer.

6. The device of claim 1 wherein at least one end portion has a threaded end for engagement with a corresponding threaded portion of a mating flow connector of the blood treatment set.

7. The device of claim 1 wherein the actuating member has a cylindrical portion attached coaxially to the exterior portion, wherein the cylindrical portion is shaped and adapted for sliding engagement with a rigid port of a medical device chamber.

8. The device of claim 1 wherein the fitting is naturally biased to the second position so that the slit is closed.

9. The device of claim 1 wherein the fitting includes at least a portion formed of polyurethane.

10. A medical valve device for use with a blood treatment set, the device comprising:
a tubular fitting including:
first and second chambers,
a first end portion for engagement and communication with a first port, the first end portion communicating with the first chamber,
a second end portion for engagement and communication with a second port, the second end portion communicating with the second chamber, and
a generally central portion having an internal flexible barrier having a slit extending between the first chamber and the second chamber; and
an actuating member in rotatable engagement radially around the fitting central portion and including:
an exterior portion for manual movement of the actuating member, and
an inner surface generally in contact with the fitting central portion, the inner surface including at least a first flat,
wherein the actuating member is moveable to a first position whereby the first flat is positioned relative to the slit such that the first and second chambers may communicate through the slit in an open position, and the actuating member is movable to a second position whereby the first flat is positioned relative to the slit such that the slit is in a closed position to prevent communication therethrough.

11. The device of claim 10 wherein the fitting provides a natural bias to the actuating member so that the actuating member is either in the first position or second position.

12. The device of claim 11 wherein the fitting slit is in a naturally closed position, and the actuating member in the second position compresses the fitting to open the slit.

13. The device of claim 10 wherein a pressure differential between the first and second chambers biases the slit to a closed position.

14. The device of claim 10 wherein the first end portion is engageable with a male end of a blood flow tubing assembly, and the second end portion is engageable with a rigid portion of a further blood treatment chamber.

15. The device of claim 10 wherein actuating member includes a second flat generally opposed to the first flat, the two flats cooperating in the first and second positions to open and close, respectively, the slit.

16. The device of claim 15 wherein the flats are joined by opposed arcuate surface portions, the flat surface portions acting as cams to compress the fitting central portion.

17. The device of claim 10 wherein the actuating member exterior portion includes a handle portion for manual actuating thereof.

18. The device of claim 10 wherein the tubular fitting is a unitary member formed integral and made of elastomer.

19. The device of claim 10 wherein at least one end portion has a threaded end for engagement with a corresponding threaded portion of a mating flow connector of the blood treatment set.

20. The device of claim 10 wherein the actuating member has a cylindrical portion attached coaxially to the exterior portion, wherein the cylindrical portion is shaped and adapted for sliding engagement with a rigid port of a medical device chamber.

21. The device of claim 10 wherein the fitting includes at least a portion formed of polyurethane.
